(19) United States
(12) Patent Application Publication
(10) Pub. No.: US 2024/0350076 A1
(43) Pub. Date: Oct. 24, 2024

(54) VITAL SIGNS BEACON SYSTEM

(71) Applicant: OWLink Technology, Inc., Irvine, CA (US)

(72) Inventors: Shing-Wu Paul Tzeng, Irvine, CA (US); Ding-Wei CHENG, New Taipei City (TW); Andrew DONG, Corona del Mar, CA (US)

(21) Appl. No.: 18/304,736

(22) Filed: Apr. 21, 2023

(51) Int. Cl. A61B 5/00; A61B 5/0205; G16H 40/67

(52) U.S. Cl. CPC A61B 5/4809; A61B 5/0002; A61B 5/0205; A61B 5/6892; A61B 5/746; G16H 40/67; A61B 5/05; A61B 5/0816; A61B 5/1102; A61B 2562/0219; A61B 2562/028

(57) ABSTRACT

Monitoring and action related to an end user's sleep activity may be provided by a system, device, and associated methods to monitor an end user's heart activity. A ballistocardiograph (BCG) signal may be detected by a physiological sensor that is not in contact with the end user. The BCG signal may be used to determine one of a plurality of sleep related statuses and health. Reports, alerts, and control of local electronic devices may be performed by a remote computer server and analysis engine that process the BCG signal.

### CROSS-REFERENCE TO RELATED APPLICATIONS

None.

### FIELD

The subject disclosure relates to network systems, and more particularly, to a vital signs beacon system.

### BACKGROUND

Sleep health, quality of sleep, and sleep related conditions such as sleep apnea are commonly monitored using wired systems such as an electrocardiogram (ECG) machine. In an ECG setup, electro-pads must be adhered to the person's skin and wires leading to the ECG machine are connected to the pads. ECG signals provide current levels of heart activity. Generally, multiple ECG wires are connected to the person restricting the person from movement while sleeping.

Sometimes a respirator or other intubation device may be connected to the person sleeping to measure respiratory activity. The person is generally restricted in their freedom of movement because the tubes have limited slack and can be somewhat inflexible.

As is understood by any who have had to wear ECG wiring or be intubated while sleeping, the act of sleeping is itself negatively impacted by being hooked up to wiring and tubes because the wires and tubes are uncomfortable, restrict movement, and the machines connected to the wires and tubes can be loud.

### SUMMARY

In one aspect of the disclosure, a system is disclosed. The system includes a monitor device coupled to a bed and positioned proximate but not in contact with an end user, and configured to monitor vital signs of the end user. The device includes a physiological sensor, a digital signal processor coupled to the physiological sensor, and a wireless radio transmitter coupled to the digital signal processor. The physiological sensor is disposed to detect a vibrational signal from a heart of the end user. The digital signal processor is configured to translate the vibrational signal into a ballistocardiograph (BCG) signal. The wireless radio transmitter is configured to transmit the BCG signal through a network. The system further includes a computing server and a sleep analysis engine resident on the computing server. The sleep analysis engine is configured to analyze the BCG signal transmitted through the network and determine a plurality of sleep statuses associated with the vital signs of the end user.

In another aspect of the disclosure, a system is disclosed. The system includes a ballistocardiograph (BCG) monitor device coupled to a bed and positioned proximate but not in contact with an end user. The BCG monitor device is configured to monitor vital signs of the end user and includes: a three axis Micro-Electro-Mechanical Systems (MEMS) based accelerometer, a first processor coupled to the three axis MEMS based accelerometer, and a wireless radio transmitter coupled to the processor. The three axis MEMS based accelerometer is disposed to detect a vibrational signal from a heart of the end user. The first processor is configured to translate the vibrational signal into a BCG signal, and the wireless radio transmitter is configured to transmit the BCG signal through a network. The system also includes a position monitor module which includes a RADAR sensor, a thermal array of sensors, and a second processor coupled to the RADAR sensor and the thermal array of sensors. The second processor is configured to process data from the RADAR sensor and from the thermal array of sensors into movement, location, and occupancy data associated with the end user. The system further includes a computing server and a sleep analysis engine resident on the computing server. The sleep analysis engine is configured to: analyze the BCG signal transmitted through the network and determine a plurality of sleep statuses associated with the vital signs of the end user; determine whether the end user has fallen asleep based on the BCG signal; determine, through the network, whether one or more electrical consumer devices in an end user's home are connected to the network; adjust by the computing server, a state of the one or more electrical consumer devices; determine, based on the BCG signal and the movement, location, and occupancy data, whether the end user has fallen; predict, based on the BCG signal and the movement, location, and occupancy data, whether the end user has or will wake up; and send an alert to a third party in response to the determination of the end user having fallen or the prediction of the end user waking up.

In still yet another aspect of the disclosure, a computer program product for detecting sleep related characteristics is disclosed. The computer program product includes one or more computer readable storage media. A set of program instructions collectively stored on the one or more computer readable storage media include detecting, by a digital signal processor, a vibrational signal from a heart of an end user sleeping in a bed, wherein the vibrational signal is detected by a physiological sensor positioned proximate and not in contact with the end user. The vibrational signal is translated into a ballistocardiograph (BCG) signal. The BCG signal is transmitted through a network to a computer server. The BCG signal transmitted through the network is analyzed. The computer server determines a plurality of sleep statuses associated with the vital signs of the end user based on the BCG signal.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

FIG. 1 is a block diagram of a system and network environment for detecting sleep characteristics of an end user, according to an example embodiment.

FIG. 2 is a block diagram of a vital signs monitor device with a thermal and position/body gesture sensor monitor device in a wireless connection environment, according to an example embodiment.

FIG. 2A is a plot comparison of ballistocardiograph and an electrocardiograph waveforms consistent with embodiments.

FIG. 2B is an illustration of aortic flow.

FIG. 3A is a front view of a vital signs monitor device, according to an example embodiment.

FIG. 3B is a right side edge view of the vital signs monitor device of FIG. 3A.

FIG. 3C is a left side edge view of the vital signs monitor device of FIG. 3A.

FIG. 3D is a rear end edge view of the vital signs monitor device of FIG. 3A.

FIG. 3E is a front end edge view of the vital signs monitor device of FIG. 3A.

FIG. 4 is a schematic illustration of the vital signs monitor device of FIG. 3A and the temperature and position sensor monitor of FIG. 2 coupled to a bed frame with an end user in the bed, consistent with embodiments.

FIG. 5 is a block diagram of consumer devices controlled wirelessly consistent with embodiments disclosed herein.

FIG. 6 is a screenshot of a user interface consistent with embodiments.

FIG. 7 is a screenshot of a dashboard for a software app consistent with embodiments.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. Like or similar components are labeled with identical element numbers for ease of understanding.

### Overview

In general, embodiments of the subject technology provide monitoring and action related to an end user's sleep activity. A system, device, and associated methods monitor an end user's heart activity to determine one of a plurality of sleep related statuses and health. Embodiments of the subject technology may monitor the end user without direct contact. For example, the device includes a physiological sensor that detects heart activity without needing to be worn while the end user sleeps. As will be appreciated by end users, the quality of sleep is not affected by the constraints associated with a worn device which may interfere with sleep. Instead, the device may be wireless and placed proximate the end user (for example, on a bed frame or headboard, and will still detect heart activity signals that the system will analyze to determine sleep status of the user. In addition, the system may determine based on analysis of the signal provided by the device whether any other devices in the user's environment should be activated or turned off, or whether any third parties need to be contacted because the end user is exhibiting uncharacteristic sleep activity.

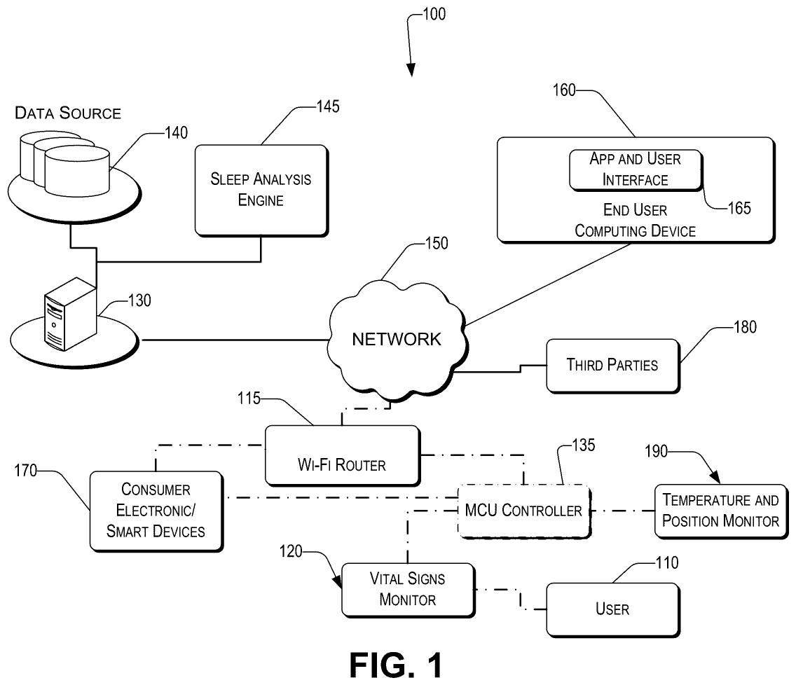

Referring now to FIG. 1, a system 100 for monitoring an end user's sleep activity is shown according to an embodiment. The system 100 includes a vital signs monitoring device 120 (referred to herein simply as the “device 120”) connected wirelessly to a network. The device 120 may be positioned proximate to, but not in contact with, the end user 110. Some embodiments may include a temperature and position monitor device 190 that generally detects changes in the position of the end user and heat from the user and surrounding environment.

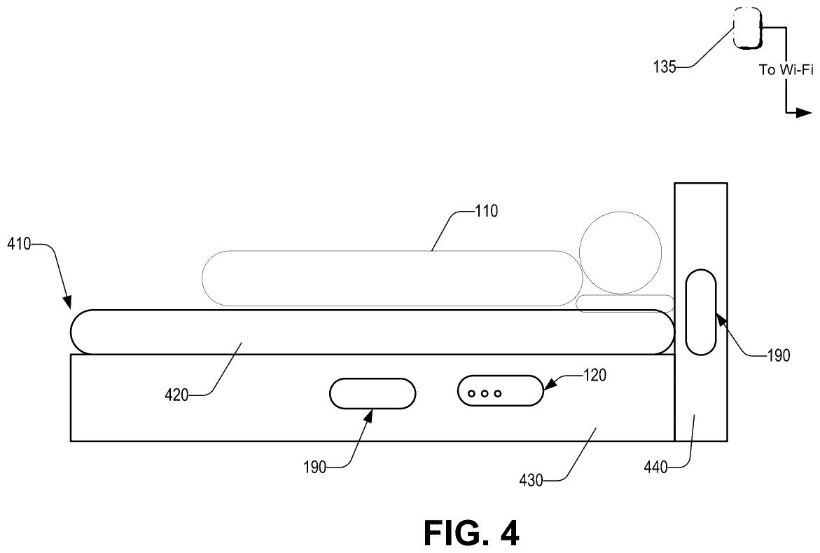

For example, and referring temporarily to FIG. 4, the device 120 is shown in an example use application for monitoring the end user 110 sleeping in a bed 410. The bed 410 may include a mattress 420, a bed frame 430, and a headboard 440. The device 120 is shown coupled to the bed frame 430 within range of the end user's heart activity. Another example placement of the device 120 may include coupling the device 120 to the headboard 440. Signals from the end user's heart may be detected by the device 120 without direct physical contact with the end user's body. Details providing an example of indirect detection are discussed further below with respect to the device 120.

Referring back now to FIG. 1, the system 100 may also include a wireless connection to a Wi-Fi router 115 within the end user's environment (for example, general place of residence) that transmits a signal from the device 120 and/or the temperature and position monitor device 190 through the network 150 to a computer server 130. Some embodiments may include a controller module 135 that may be configured to act as an intermediary device transmitting data and signals from the device 120 and/or the temperature and position monitor device 190 to the Wi-Fi router 115. The controller module 135 may be wirelessly or connected to or hardwire connected to the device 120 and/or 190. The controller module 135 may include a processor unit that may be configured to coordinate the signals from the devices 120 and 190 for re-transmission. While the controller module 135 is shown intermediate the Wi-Fi router 115 and the devices 120 and 170, in some embodiments the devices 120 and 170 may communicate directly through the Wi-Fi router 115 to the network 150.

The computer server 130 may include a data source 140 and a sleep analysis engine 145. The data source 140 may include stored data on the end user's heart activity, sleep data, tables of heart activity and sleep data correlations, heart activity and sleep data for a population of users, and ancillary data associated with the end user 110 and other devices in the end user's environment that the system 100 has permission to access.

The sleep analysis engine 145 may include software programs that analyze real-time physiological statuses of the end user 110, monitor a current state of the end user 110, and notify the end user 110 or third parties 180 of abnormal physiological or atypical events.

For example, the sleep analysis engine 145 may use the signal transmitted by the device 120 to determine a number of statuses related to the end user 110. The sleep analysis engine 145 may determine whether the end user 110 is in bed. The sleep analysis engine 145 may determine a current heart rate, respiration rate, and heart variability rate of the end user 110. The sleep analysis engine 145 may determine whether the end user 110 has entered into a sleep state. The sleep analysis engine 145 may analyze the device 120 signal to determine a quality of sleep for the night. The sleep analysis engine 145 may analyze the device 120 signal to determine sleep patterns and routines of the end user 110. The sleep analysis engine 145 may analyze the device 120 signal to determine vital sign boundaries and threshold values that indicate variations in vital signs that warrant notifying a third party 180. The sleep analysis engine 145 may also dynamically generate various physiological statistical reports (daily, weekly, and monthly) for observation and analysis during the period. The reports may be useful to the end user 110 and the third parties 180 in order to facilitate the use of smart health management based on various physiological changes that can be seen via the reports. The reports may be accessed by the end user 110 or third parties 180 using an app and user interface (UI) 165 on a computing device 160. Additionally, settings for triggering notifications (such as alerts of atypical activity of the end user 110 to third parties 180) may be set through the app and UI 165.

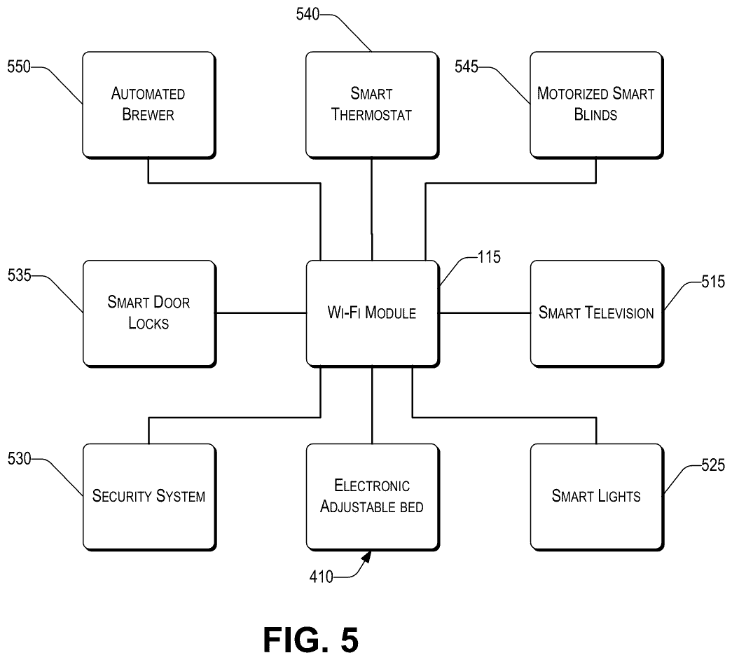

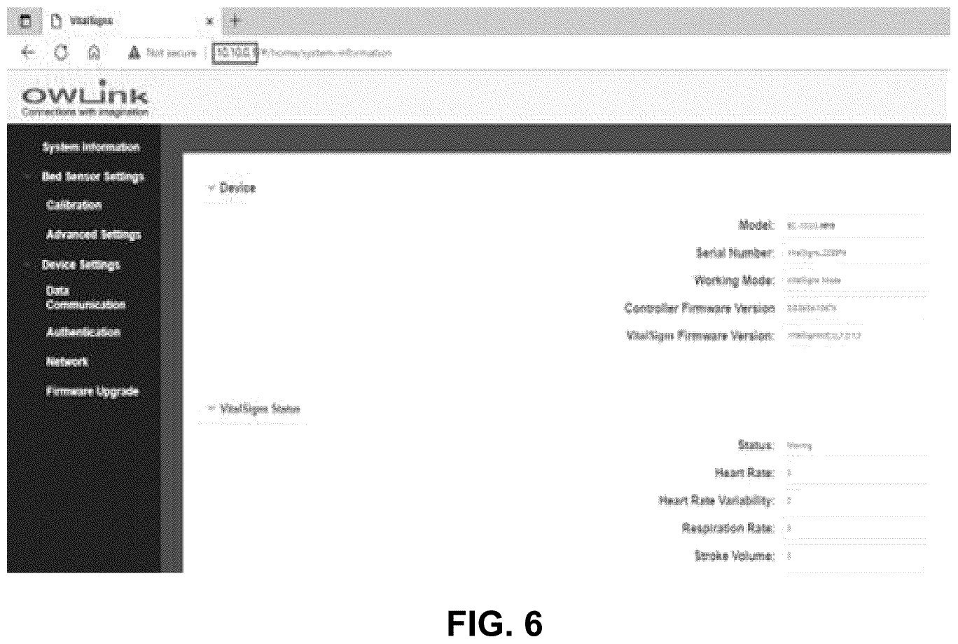

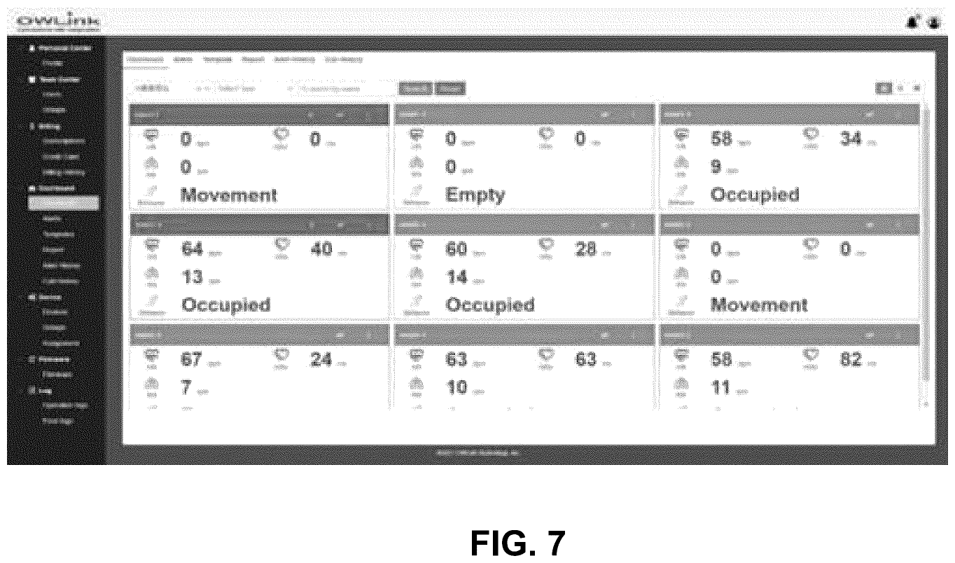

In some embodiments, the system 100 may provide automated control of consumer devices 170 in the end user's environment. The computing server 130 may control the consumer devices 170 in the end user's environment remotely based on the end user's statuses as determined by the sleep analysis engine 145 based on the signal from the device 120. In some embodiments, the consumer devices 170 may be smart devices. As can be seen in FIGS. 1 and 5, the consumer devices 170 may be accessible directly through the Wi-Fi router 115 or through the controller module 135 for automated control in the end user's environment. When a direct connection through the Wi-fi router 115 is used, the control signals may be provided by the server 130. When the connection to consumer devices 170 is routed through the controller module 135, the processor unit in the controller module 135 may control the consumer devices 170. For example, when the computer server 130 has determined that the end user 110 has fallen asleep (in some instances, involuntarily while watching a TV), the computer server 130 may determine the person's sleep state and sends a signal to turn off the smart TV 515. Detecting that the person is asleep may sometimes trigger the computer server 130 to turn off smart lights 525 (either in the same room as the end user 110 or including other rooms). Some embodiments of the computer server 130 may turn on the user's security system 530 when the end user 110 is found to be asleep. Some embodiments may control an electronic/smart door lock 535 when the user is asleep by locking the door. Some embodiments may adjust electronic/smart thermostats 540 to a programmed temperature when the user is asleep. If the end user 110 owns electronic blinds 545, the computer server 130 may close one or more sets of window blinds to eliminate light in the user's room and improve sleep quality. If the bed 410 (FIG. 4) is adjustable and electronic, the computer server 130 may control the bed 410 to move into a previously set sleep position including the bed height.

In some embodiments, the sleep analysis engine 145 may determine when a person is about to wake up based on a current signal from the device 120. In response to the computer server 130 determining that the end user 110 will awake within a threshold time window (for example, 5 minutes), the computer server 130 may remotely control the lights 525 to activate (and in some embodiments to a gradual light intensity). The computer server 130 may control the window blinds 545 to open. The computer server 130 may control the smart/electronic thermostat(s) 540 to raise or lower the temperature to a user programmed comfort level. The computer server 130 may control the adjustable bed 410 to a previously set wake up position. In some embodiments, the computer server 130 may be wirelessly connected to an automatic brewer machine 550 (for example, that brews coffee or tea), and may control the brewer 550 to start brewing a beverage when detecting the end user's proximity to waking up or that the end user has woken up.

When a person is falling asleep in a bed position that is not the same as the previously set sleep position, the system will determine the person's (user 110) sleep state and may turn off lights 525, turn on security system 530, trigger the lock 535 to lock the door, adjust thermostats 540, window blinds 545, and bed 410 to the previously set sleep position.

In some embodiments, the computer server 130 may determine abnormal or atypical activity associated with the end user 110 and may alert third parties 180 of the situation so that the third parties may for example, check on the welfare of the end user 110. Third parties may include for example, family, friends, neighbors, caretakers, administrative staff in a housing facility or hospital, or emergency services. For example, when the computer server 130 determines that the end user 110 has an unexpected heart rate or the user's the heart rate drops below the average and a standard deviation of values for some period of time, the computer server 130 may send an alert to third parties 180. When the computer server 130 determines that the end user 110 has an unexpected respiration rate or the user's the respiration rate drops below the average and a standard deviation of values for some period of time, the computer server 130 may send an alert to third parties 180.

In some embodiments, the computer server 130 may detect that the end user 110 is not in the bed 410 (FIG. 4) at an expected time and within a threshold variation value from the expected time; for example, the bed is not occupied during 11 PM to 2 AM when the end user 110 is typically occupying the bed between 9 PM to 10 PM. An alert may be sent to third parties 180 indicating the user's absence from bed. This may be helpful for example, in signaling that the end user is incapacitated and unable to make it to bed.

In some embodiments, the computer server 130 may detect that the end user 110 is in the bed 410 (FIG. 4) at an unexpected time within a variation of time; for example, the bed is occupied during 10 AM to 12 PM when the end user 110 is typically out of the bed between 8 AM to 9 AM. An alert may be sent to third parties 180 indicating the user's atypical occupancy of the bed. This may be helpful for example, in signaling that the end user may be ill and unable to get out of bed.

In some embodiments, the sleep analysis engine 145 may determine when the end user's sleep patterns or routines deviate beyond an acceptable variation of activity; for example, the end user has gotten out of bed 6 times during a sleep session when the person typically gets out of bed only 2 times. The computer server 130 may send an alert to third parties 180 indicating the atypical behavior.

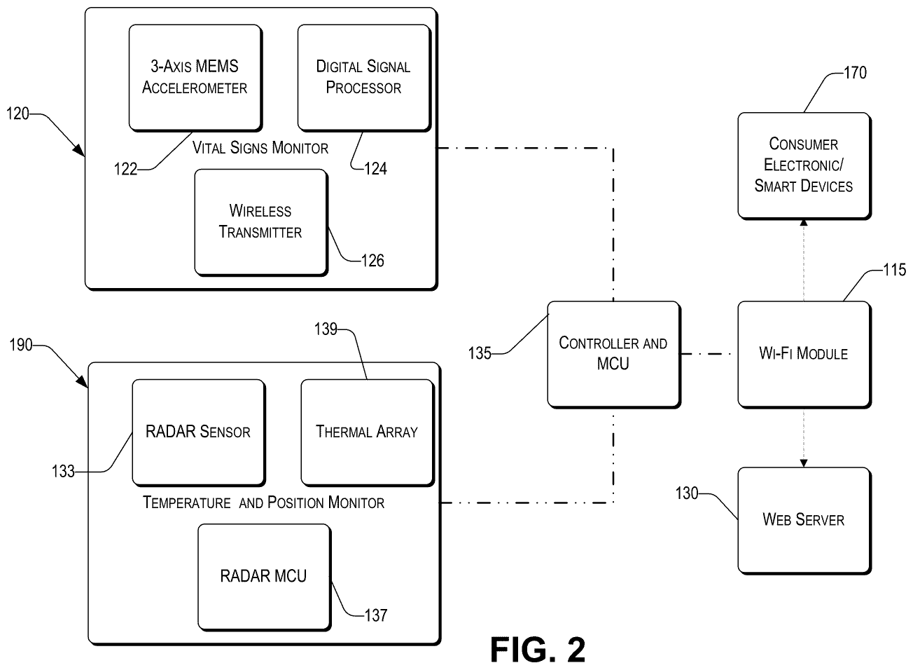

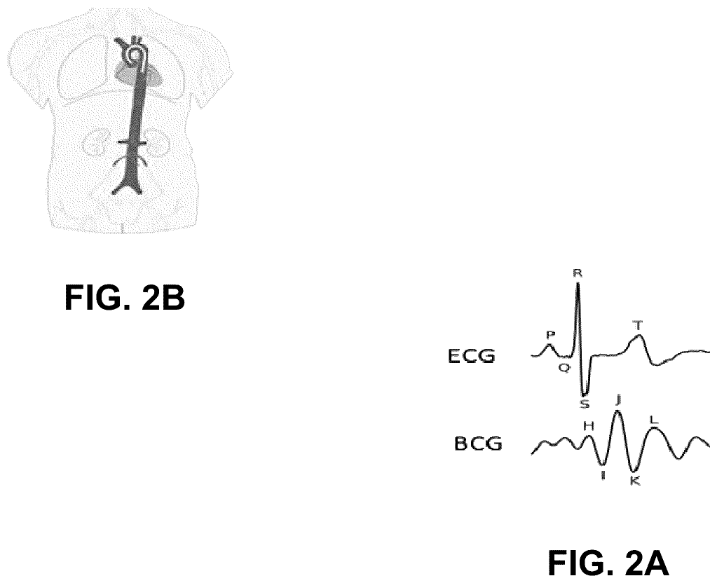

FIG. 2 shows an architecture of the device 120 and the device 190 in further detail. The device 120 may include a physiological sensor 122, a digital signal processor 124, and a wireless transmitter 126. The physiological sensor 122 may be for example, a three-axis Micro-Electro-Mechanical Systems (MEMS) based accelerometer. The physiological sensor 122 captures ballistocardiograph (BCG) vibrations generated by activity of the end user's heart (See FIG. 2A). A BCG vibration is detectable by the sensor 122 because when a user is in the bed, whenever the heart beats (See FIG. 2B for an illustration of aortic activity), after the aortic valve opens, blood accelerates from the heart to the head, with an equal and opposite rebound force H→I generated. Then the aorta will turn downward, and the blood flow acceleration will be reversed I→J. When the blood flows through the artery, the acceleration changes the force of direction J→K almost simultaneously with the closure of the aortic valve. The rest of the signal will be varied by the damping factor and the pressure and kinetic energy created by the blood flowing in the blood vessel, but it will gradually weaken K→L before the next heartbeat occurs.

As blood flows from the human heart into the aorta, the aorta turns and the blood pressure pulse continues to enter the arteries, causing the recoil force to cause weak vibrations throughout the body. In embodiments using an accelerometer, even weak vibration signals may be captured by ultra-high-sensitivity accelerometers, and then filtered and extracted from various physiological signals such as heartbeat and respiration data through the algorithm programmed into the digital signal processor 124. The DSP 124 converts the output data of the physiological sensor 122 into data that represents heart rate (Heart Rate), respiratory rate (Respiratory Rate), blood flow Impact strength (Stroke Volume) and bed (Occupied) or out of bed (Empty) and other data. The DSP 124 data is forwarded by the wireless transmitter 126 to the network 150 and on to the computer server 130 (FIG. 1) for analysis. As may be appreciated, the device 120 does not need to be electrically connected or in physical contact with the end user yet provides data that can be used to analyze sleep quality and alert the user or third parties of abnormal activity.

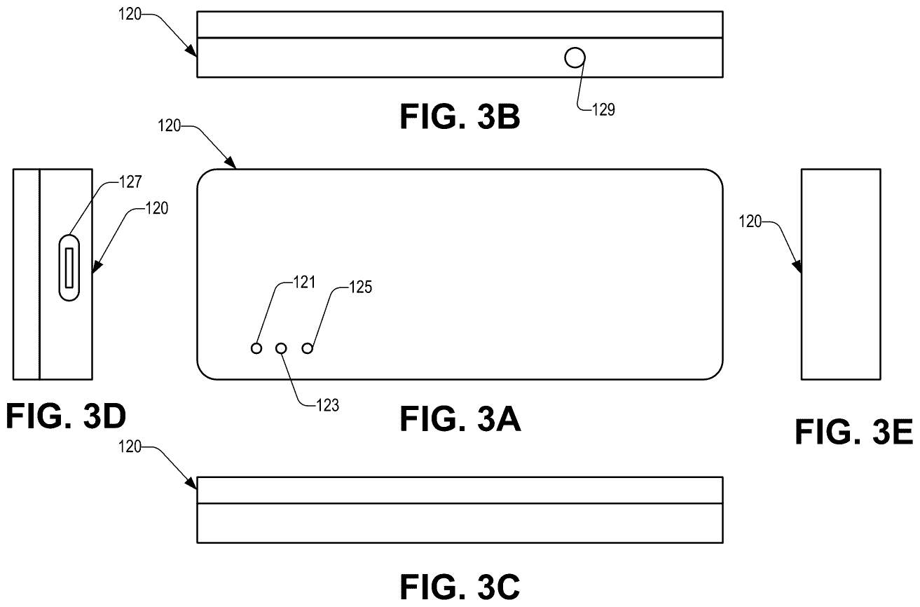

FIGS. 3A-3E show various external views of the device 120. The device 120 may include indicator lights 121 (for showing a current power on/off state), 123 (for showing a current Internet connection status), and 125 (for showing whether physiological data is transmitting). Some embodiments may include a connection port 127 which may be used to receive power and/or data (for example, a USB port). Some embodiments may include a default reset button 129 to restore the device 120 settings to a factory reset condition.

The temperature and position monitor device 190 may include a RADAR sensor 133 (for example, a 60 GHz sensor) and a RADAR processor 137 that determines when movement above a threshold value has occurred. In some embodiments, the device 190 does not include a processor 137 and the processing of RADAR and/or thermal signals may be performed by the processor in the controller module 135. In some embodiments, the device 190 may include a thermal array 139 of sensors that detects heat levels around the end user. The RADAR sensor 133 and/or the thermal array 139 detect motion of the end user (or of others in the room). The processor 137 may be configured to analyze the motion and determine whether different types of movement. For example, the temperature and position monitor device 190 may register people count, motion, occupancy of the room or bed, and movements of the user getting in or out of bed or falling down. Data from the processor 137 may be sent wirelessly (either through a dedicated wireless transmitter in the device 190 or by a wireless transmitter shared with the device 120) to the server 130 and sleep analysis engine 145 (FIG. 1) to analyze and predict when an end user will or has gotten out of bed. Analysis of the data may be used to predict, prevent, and/or detect falls by the end user. Detected falls include for example, falls from the bed or falls outside of the bed. In another embodiment, person location and movement may be tracked. In some applications, the prediction and/or detection may be sent to third parties that watch over or are providing in-house care of the end user. If any monitoring value goes over/under a threshold value for a threshold time, an alert message may be sent to third parties 180 (FIG. 1), smartphones or any other connected devices.

FIG. 5 shows an example user interface that may be accessed through the app 165 (FIG. 1). The UI shows for example, device specific identification numbers and end user current vital sign data as detected by the BCG signal from the device 120 (FIG. 1).

FIG. 6 shows an example of another UI that displays in a dashboard format, real-time end user heart activity data provided by the computer server 130. Data such as heart rate, heart rate pumping velocity, respiratory rate, and behavior indictors (for example, bed occupancy) are shown.

As will be understood, some embodiments of the subject technology are provided by computer implemented methods and computing devices. It will be understood that a computing device may serve different roles depending on the need in the system or depending on the step being performed in a process. For example, in the role of a web server, a host server, or an online platform server, a computing device may implement for example the functions related to backend processes (for example, administering the analysis of heart activity, generation of reports to an app and UI, and remote control of end user environmental device). In another role, the computing device may be a client device that includes communication functions operated by end users to interact with the system (for example, devices 160 shown in FIG. 1). In the role of a user device, the computing device is generally not a server but may instead be desktop computers, tablet or laptop computers, all-in-one computer stations, a mobile computing device (for example, a smart phone, smart wearable devices (glasses, jewelry, watches, car wear, etc.), smart televisions, smart hubs, robots, or programmable electronics. As will be understood, the end user device may generally provide frontend aspects of the system. In some embodiments however, the frontend computing device may perform one or more of the backend steps where possible.

The computing device generally includes a computer program product having a set of program modules including files and executable instructions, and a bus system that couples various system components including system memory to the processor(s). The memory storage may store for example, user profiles, historical user data, threshold values for triggering alerts, and password or general access data for controlling local environmental devices of the end user. The program modules generally carry out the functions and/or methodologies of embodiments as described above. The computing device may typically include a variety of computer system readable media. Such media could be chosen from any available media that is accessible by the computing device, including non-transitory, volatile and non-volatile media, removable and non-removable media for use by or in connection with an instruction execution system, apparatus, or device. The system memory could include one or more computer system readable media in the form of volatile memory, such as a random-access memory (RAM) and/or a cache memory. Some embodiments may generate an electronic user interface (viewable and controllable from the display unit) that may allow the user to control automated device controls upon sleep or wake-up, view reports, set threshold values, and notification related data.

As will be appreciated by one skilled in the art, aspects of the disclosed invention may be embodied as a system, method or process, or computer program product. Accordingly, aspects of the disclosed invention may take the form of an entirely hardware embodiment, an entirely software embodiment (including firmware, resident software, micro-code, etc.) or an embodiment combining software and hardware aspects that may all generally be referred to herein as a “module”, “circuit”, or “system.” Furthermore, aspects of the disclosed invention may take the form of a computer program product embodied in one or more computer readable media having computer readable program code embodied thereon.

Aspects of the disclosed invention are described above with reference to block diagrams of methods, apparatus (systems) and computer program products according to embodiments of the invention. It will be understood that each block of the block diagrams, and combinations of blocks in the flowchart illustrations and/or block diagrams, can be implemented by computer program instructions. These computer program instructions may be provided to the processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks in the figures.

Those of skill in the art would appreciate that various components and blocks may be arranged differently (e.g., arranged in a different order, or partitioned in a different way) all without departing from the scope of the subject technology. The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects.

Thus, the claims are not intended to be limited to the aspects shown herein, but are to be accorded the full scope consistent with the language of claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

Terms such as “top,” “bottom,” “front,” “rear,” “above,” “below” and the like as used in this disclosure should be understood as referring to an arbitrary frame of reference, rather than to the ordinary gravitational frame of reference. Thus, for example, an item disposed above another item may be located above or below the other item along a vertical, horizontal or diagonal direction; and an item disposed below another item may be located below or above the other item along a vertical, horizontal or diagonal direction. Some frames of references and the position of claimed structure relative to those frames of reference may be gleaned from the appended drawings. Yet, it should be understood that for some embodiments, the detection from devices disclosed herein may not be restricted to being accomplished from any particular position shown, whether the device is above, below, or on one side of the user being monitored.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A system, comprising:
a monitor device coupled to a bed and positioned proximate but not in contact with an end user, and configured to monitor vital signs of the end user, including:
a physiological sensor;
a processor coupled to the physiological sensor; and
a wireless radio transmitter coupled to the processor, wherein:
the physiological sensor is disposed to detect a vibrational signal from a heart of the end user,
the processor is configured to translate the vibrational signal into a ballistocardiograph (BCG) signal, and
the wireless radio transmitter is configured to transmit the BCG signal through a network; and
a computing server and a sleep analysis engine resident on the computing server, wherein the sleep analysis engine is configured to:
analyze the BCG signal transmitted through the network and determine a plurality of sleep statuses associated with the vital signs of the end user;
determine whether the end user has fallen asleep based on the BCG signal;
determine, through the network, whether one or more electrical consumer devices in an end user's home are connected to the network; and
adjust by the computing server, a state of the one or more electrical consumer devices.

2. The system of claim 1, further comprising a RADAR sensor module positioned proximate to the bed and coupled to the wireless radio transmitter, send RADAR sensor information to the processor, wherein the processor is configured to determine an occupancy, getting in or out of bed, or fall of the end user.

3. The system of claim 1, further comprising a computing device and a software application resident on the computing device, wherein the software application includes one or more user interfaces displaying reports showing the sleep statuses.

4. The system of claim 1, wherein one of the electrical consumer devices is a television wirelessly connected to the network, and the computing server turns the television off in response to determining the end user being asleep.

5. The system of claim 1, wherein:
one of the electrical consumer devices is a light wirelessly connected to the network, and the computing server turns the light off in response to determining the end user being asleep;
the computing server determines that the end user will wake up within a proximate time frame of detecting a change in sleep status; and
the computing server sends an activation signal to turn on the previously turned of light.

6. The system of claim 1, wherein: one of the electrical consumer devices is a home security system wirelessly connected to the network, and the computing server turns the home security system on in response to determining the end user being asleep.

7. The system of claim 1, wherein one of the electrical consumer devices is an electronic door lock wirelessly connected to the network, and the computing server locks the electronic door lock in response to determining the end user being asleep.

8. The system of claim 1, wherein:
one of the electrical consumer devices is a set of electronic window blinds wirelessly connected to the network, and the computing server closes the set of electronic window blinds in response to determining the end user being asleep;
the computing server determines that the end user will wake up within a proximate time frame of detecting a change in sleep status; and
the computing server sends an activation signal to turn open the previously closed set of electronic window blinds.

9. The system of claim 1, wherein one of the electrical consumer devices is an electronic thermostat wirelessly connected to the network, and the computing server adjusts the temperature of the thermostat to a first setting in response to determining the end user being asleep;
the computing server determines that the end user will wake up within a proximate time frame of detecting a change in sleep status; and
the computing server sends an activation signal to adjust the temperature of the thermostat to a second setting.

10. The system of claim 1, wherein:
one of the electrical consumer devices is an electronic brewer wirelessly connected to the network;
the computing server determines that the end user will wake up within a proximate time frame of detecting a change in sleep status;
the computing server sends an activation signal to the electronic brewer to start a brew process.

11. The system of claim 1, wherein the bed is an adjustable bed including a controller wirelessly connected to the network, and the computing server adjusts a position of the bed in response to a sleep status of the end user being asleep, based on the BCG signal.

12. The system of claim 1, wherein the computing server is configured to:
determine when the end user is exhibiting a current hear rate based on the BCG signal, below an average heart rate and a standard deviation of values for a threshold window of time; and
send an alert signal to a third party in response to determining the end user's heart rate is below the average heart rate.

13. The system of claim 1, wherein the computing server is configured to:
determine when the end user is exhibiting a current respiration rate based on the BCG signal, below an average respiration rate and a standard deviation of values for a threshold window of time; and
send an alert signal to a third party in response to determining the end user's respiration rate is below the average respiration rate.

14. The system of claim 1, wherein the computing server is configured to:
determine, based on historical data, hours when the end user is typically in the bed;
determine whether the end user is not in bed within the hours the end user is typically in the bed, based on the BCG signal; and
send an alert signal to a third party in response to determining the end user is not in bed within the hours the end user is typically in the bed.

15. The system of claim 1, wherein the computing server is configured to:
determine, based on historical data, hours when the end user is typically in the bed;
determine whether the end user is in bed outside of the hours the end user is typically in the bed, based on the BCG signal; and
send an alert signal to a third party in response to determining the end user is in bed outside of the hours the end user is typically in the bed.

16. The system of claim 1, wherein the computing server is configured to:
determine, based on historical data, sleep patterns associated with the end user;
determine whether the end user is exhibiting sleep patterns outside a threshold variation value, based on the BCG signal; and
send an alert signal to a third party in response to determining the end user is exhibiting sleep patterns outside a threshold variation value.

17. The system of claim 1, wherein the computing server is configured to determine
a quality of sleep value for the end user based on the BCG signal, wherein the quality of sleep value is based on a combination of:
a time into the bed for the end user,
a time out of the bed for the end user,
a detected number of times the end user got out of the bed,
an onset latency of entering sleep for the end user,
an amount of wake time after entering sleep for the end user.

18. The system of claim 1, wherein physiological sensor is a three axis Micro-Electro-Mechanical Systems (MEMS) based accelerometer.

19. A system, comprising:
a ballistocardiograph (BCG) monitor device coupled to a bed and positioned proximate but not in contact with an end user, and configured to monitor vital signs of the end user, including:
a three axis Micro-Electro-Mechanical Systems (MEMS) based accelerometer;
a first processor coupled to the three axis MEMS based accelerometer; and
a wireless radio transmitter coupled to the processor, wherein:
the three axis MEMS based accelerometer is disposed to detect a vibrational signal from a heart of the end user,
the first processor is configured to translate the vibrational signal into a BCG signal, and
the wireless radio transmitter is configured to transmit the BCG signal through a network;
a position monitor module, including:
a RADAR sensor;
a thermal array of sensors;
a second processor coupled to the RADAR sensor and the thermal array of sensors, configured to process data from the RADAR sensor and from the thermal array of sensors into movement, location, and occupancy data associated with the end user; and
a computing server and a sleep analysis engine resident on the computing server, wherein the sleep analysis engine is configured to:
analyze the BCG signal transmitted through the network and determine a plurality of sleep statuses associated with the vital signs of the end user;
determine whether the end user has fallen asleep based on the BCG signal;
determine, through the network, whether one or more electrical consumer devices in an end user's home are connected to the network;
adjust by the computing server, a state of the one or more electrical consumer devices;
determine, based on the BCG signal and the movement, location, and occupancy data, whether the end user has fallen;
predict, based on the BCG signal and the movement, location, and occupancy data, whether the end user has or will wake up; and
send an alert to a third party in response to the determination of the end user having fallen or the prediction of the end user waking up.

20. A computer program product for detecting sleep related characteristics, the computer program product comprising a non-transitory computer readable storage medium having computer readable program code embodied therewith, the computer readable program code being configured, when executed by a processor, to:
detect, by a digital signal processor, a vibrational signal from a heart of an end user sleeping in a bed, wherein the vibrational signal is detected by a physiological sensor positioned proximate and not in contact with the end user;
translate, by the digital signal processor, the vibrational signal into a ballistocardiograph (BCG) signal;
transmit, by a wireless radio transmitter coupled to the digital signal processor, the BCG signal through a network to a computer server;
analyze, by the computer server, the BCG signal transmitted through the network; and
determine, by the computer server, a plurality of sleep statuses associated with the vital signs of the end user based on the BCG signal.
